(19) United States
(12) Patent Application Publication
(10) Pub. No.: US 2006/0263789 A1
(43) Pub. Date: Nov. 23, 2006

(54) Unique identifiers for indicating properties associated with entities to which they are attached, and methods for using

(76) Inventor: Robert Kincaid, Half Moon Bay, CA (US)

Correspondence Address: AGILENT TECHNOLOGIES INC., INTELLECTUAL PROPERTY ADMINISTRATION, M/S DU404, P.O. BOX 7599, LOVELAND, CO, 80537-0599, US

(21) Appl. No.: 11/133,120

(22) Filed: May 19, 2005

(51) Int. Cl. C12Q 1/68; G06F 19/00

(52) U.S. Cl. 435/6; 702/20; 977/924

(57) ABSTRACT

Methods, systems and computer readable media for sequencing a biopolymer specimen and tracking a source from which the specimen was derived. Methods, systems and computer readable media for multiplex sequencing biopolymer samples. Methods, systems and computer readable media for efficiently sequencing biopolymeric specimens through a high-throughput sequencer. Methods, systems and computer readable media for performing ratio-based analysis with a high throughput sequencer.

### BACKGROUND OF THE INVENTION

DNA and/or RNA can be detected or identified by sequencing techniques that are currently known. (Hereinafter, for simplicity, DNA refers to both DNA and RNA.) As used herein, “sequencing in reference to DNA may include determination of partial as well as full sequence information of DNA. It may also include sequence comparisons, fingerprinting, and like levels of information about a target DNA strand or segment, as well as the express identification and ordering of nucleotides in the target DNA. Several methods have been developed to sequence DNA.

The Sanger method, as described in “DNA sequencing with chain-terminating inhibitors,” Proceedings of the National Academy of Sciences, U.S.A., 74, 12, 5463-5467, is in common use for DNA sequencing and typically requires two working days and approximately 10¹⁰ nucleic acid fragments to produce a detectable band by gel electrophoresis. Gel electrophoresis is a technique to separate a mixture of digested DNA fragments. By applying an electric field to the negatively charged DNA fragments through a porous gel, the mixture of DNA fragments is separated into bands, each containing DNA fragments of the same size. Then, the base sequences of the separated DNA fragments are read from an autoradiogram of the four lanes, each lane corresponding to one of the four bases.

A major problem for this method is obtaining sufficient quantities of the substance of interest. Conventional molecular cloning (genetic engineering) techniques may be applied in an attempt to address this problem, however, such cloning techniques may introduce contamination due to the amplification of unintended DNA sequences.

Another sequencing technique, sometimes referred to as the nanopore method, applies an electric field to move nucleic acid molecules through a single nanopore. As the diameter of the nanopore is very narrow and restrictive, DNA molecules are translocated as single strands, and move through the pore in a strictly linear manner. As a DNA strand passes through a nanopore, the shape and electrical properties of each base on the strand can be monitored. As these properties are unique for each of the four bases that make up the DNA strand, scientists can use the passage of a DNA strand through a nanopore to decipher the encoded information on that strand, including errors in the code known to be associated with genetic disorders, such as cancer, for example

The nanopore techniques are very linear, as noted and typically process only a single sample at a time so that the identified sequences are properly correlated with the sample from which they originated. Accordingly, procedures for such identification processes must be closely monitored to ensure that no contamination of the sample currently being sequenced occurs.

Nanopore techniques have been used for analyte detection, see U.S. Pat. No. 6,465,193 and U.S. Publication No. 2002/0142344 A1, wherein a sample is assayed for the presence of an analyte of interest. A sample to be assayed is contacted with a targeted molecular bar code having a specific binding pair member that is specific for the analyte of interest. Following contact, the resultant mixture is incubated under conditions and for a time sufficient to allow binding of the targeted bar codes to the specific analyte, if present in the sample. Following complex formation resulting from the incubation, any unbound targeted molecular bar code material is separated from the complexes. After separation of unbound targeted molecular bar code material, the molecular bar code of the analyte/targeted molecular bar code complex is separated from the remainder of the complex, i.e., the specific binding pair member and the analyte. The molecular bar codes are then detected, using any convenient protocol and are then related to the presence of the analyte of interest in the sample which the read bar code is specific to. Nanopore techniques are one such detection protocol that may be employed.

There is a continuing need for better and improved techniques to increase the speed and accuracy of sequencing. There are continuing needs for improved techniques and protocols for making it more convenient to mass process samples for sequencing, while lessening risks of contamination.

### SUMMARY OF THE INVENTION

Methods, systems and computer readable media are provided for sequencing a biopolymer specimen and tracking a source from which the specimen was derived. The biopolymer specimen may be processed to associate a unique identifier therewith, wherein the unique identifier represents metadata identifying a source sample from which the biological specimen was taken. The unique identifier may be configured to form a unique, repeatable, characteristic signature when read by a high-throughput sequencer. The biopolymer specimen with the associated unique identifier is passed through the high-throughput sequencer so that a sequence of the biopolymer specimen is identified, and the unique identifier is also identified as each passes through the high-throughput sequencer. The identified sequence of the biopolymer specimen is correlated with the source sample from which the identified sequence was derived, based upon the identifier metadata derived from the identification of the unique identifier for that respective sequence.

Methods, systems and computer readable media are provided for multiplex sequencing biopolymer samples, including processing biopolymer strands in a first biopolymer sample to provide a first unique identifier with each biopolymer strand so processed, wherein the first unique identifier includes metadata identifying the first biopolymer sample, and the first unique identifier is configured to form a unique, repeatable, characteristic signature when read by a high-throughput sequencer; processing biopolymer strands in a second biopolymer sample to provide a second unique identifier with each second biopolymer so processed, wherein the second unique identifier includes metadata identifying the second biopolymer sample, and the second unique identifier is configured to form a unique, repeatable, characteristic signature different from the signature formed by the first unique identifier, when read by the high-throughput sequencer; mixing together processed strands of the first biopolymer sample associated with the first unique identifier, with processed strands of the second biopolymer sample associated with the second unique identifier; randomly passing at least one processed strand through at least one high-throughput sequencer and identifying the strand sequence, as well as identifying the unique identifier associated therewith, as each processed strand passes through the high-throughput sequencer, respectively; and correlating the identified sequences of the biopolymers with the samples from which they were derived, based upon the identifier metadata derived from the identification of the unique identifier associated with that respective biopolymer strand.

Methods, systems and computer readable media are provided for efficiently sequencing biopolymeric specimens through a high-throughput sequencer, including processing sequences in a first biopolymeric sample to provide a first unique identifier with each processed sequence, wherein the first unique identifier represents metadata identifying said first biopolymeric sample, and the first unique identifier is configured to form a unique, repeatable, characteristic signature when read by a high-throughput sequencer; passing the sequences having first unique identifiers associated therewith through the high-throughput sequencer and identifying each sequence of the first biopolymeric sample as well as identifying the first unique identifier associated therewith, as each passes through the high-throughput sequencer; correlating the identified sequences with the first biopolymeric sample from which the identified sequences were derived, based upon the identifier metadata derived from the identification of the first unique identifier for each respective sequence; processing sequences in a second biopolymeric sample to provide a second unique identifier with each process sequence from said second biopolymeric sample, wherein the second unique identifier represents metadata identifying the second biopolymeric sample, and the second unique identifier is configured to form a unique, repeatable, characteristic signature when read by a high-throughput sequencer; passing the sequences having second unique identifiers associated therewith through the high-throughput sequencer and identifying each sequence, as well as identifying the second unique identifier associated therewith, as each passes through the high-throughput sequencer; and correlating the identified sequences with the second biopolymeric sample from which the identified sequences were derived, based upon the identifier metadata derived from reading the second unique identifier for each respective sequence, but ignoring the identified sequences when the associated unique identifier read is not the second unique identifier, or there is no unique identifier associated with the sequence.

Methods, systems and computer readable media are provided for efficiently sequencing biopolymeric specimens through a high-throughput sequencer, including processing sequences in at least one biopolymeric sample to provide a unique identifier with each sequence so processed, wherein the unique identifiers with respect to each sample are unique from unique identifiers with respect to all other samples and each unique identifier represents metadata identifying the biopolymeric sample from which each sequence associated with each unique identifier was taken from, and each unique identifier is configured to form a unique, repeatable, characteristic signature when read by a high-throughput sequencer; passing the sequences having associated unique identifiers through the high-throughput sequencer and identifying each sequence, as well as identifying any unique identifier associated therewith, as each passes through the high-throughput sequencer; and correlating the identified sequences with the respective biopolymeric samples from which the identified sequences were derived, based upon the identifier metadata derived from the identification of the associated unique identifier for each respective sequence, but ignoring the identified sequences when the associated unique identifier read is not a unique identifier associated by the processing step, or when there is no unique identifier associated with the sequence.

Methods, systems and computer readable media are provided for performing ratio-based analysis with a high throughput sequencer, including processing sequences in a test biopolymeric sample to associate a first unique identifier with each sequence so processed, wherein the first unique identifier represents metadata identifying the test biopolymeric sample, and the first unique identifier is configured to form a unique, repeatable, characteristic signature when read by a high-throughput sequencer; processing sequences in a control biopolymeric sample to associate a second unique identifier with each sequence from the control sample so processed, wherein the second unique identifier represents metadata identifying the control biopolymeric sample, and the second unique identifier is configured to form a unique, repeatable, characteristic signature different from the signature formed by the first unique identifier, when read by the high-throughput sequencer; mixing together processed sequences of the test biopolymeric sample and the first unique identifier, with processed sequences of the control biopolymeric sample and the second unique identifier; randomly passing processed sequences through at least one high-throughput sequencer and identifying the sequences, as well as identifying the unique identifiers as the processed sequences pass through a high-throughput sequencer, respectively; correlating the identified sequences with the samples from which they were derived, based upon the identifier metadata derived from the identification of the unique identifier associated with that respective sequence; counting the number of times that a particular sequence is read with regard to the first and second unique identifiers; and calculating a ratio comparing the number of times that the particular ratio was identified as associated with the first and second identifiers, respectively.

The present invention also encompasses forwarding, transmitting and/or receiving results from any of the methods described herein.

These and other advantages and features of the invention will become apparent to those persons skilled in the art upon reading the details of the systems, methods and computer readable media as more fully described below.

### BRIEF DESCRIPTION OF THE DRAWINGS

FIG. 1 is a schematic representation of an individual nucleic acid molecules being moved through a nanopore.

FIG. 2 is a flowchart illustrating events that may be carried out according to an embodiment of the present invention.

FIG. 3 schematically illustrates steps that may be performed for bi-directional sequencing of PCR products using tailed-primers in accordance with one embodiment of the present invention.

FIG. 4 is a flowchart illustrating events that may be carried out according to an embodiment of the present invention.

FIG. 5 illustrates a typical computer system that may be employed in accordance with an embodiment of the present invention.

### DETAILED DESCRIPTION OF THE INVENTION

Before the present methods, systems and computer readable media are described, it is to be understood that this invention is not limited to particular barcodes, sequences, hardware, software, step or steps described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a barcode” includes a plurality of such barcodes and reference to “the nanopore” includes reference to one or more nanopores and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Definitions

An “identifier” or “unique identifier”, as used herein, refers to an entity used to tag a biopolymer. Such entity may be a unique barcode identifier in the form of an additional unique sequence of nucleic acids appended to a nucleic acid sequence that is being tagged. Alternatively, such an identifier may be any other entity that is configured to be translocated through a nanopore and that generates a modulated signal to form a unique, repeatable, characteristic signature identifying the identifier as unique from other identifiers. Other forms of candidates for unique identifiers that may be employed, and are typically charged, include block copolymers that may comprise synthetic nucleic acids (SNAs), or other non-nucleic acid polymers suitable for detection by a nanopore sequencer.

“Metadata” refers to any information that is useful to track along with the sample/DNA strand or other sequence-based sample that is being processed. Examples of metadata include, but are not limited to: lab protocols used for the associated sample/DNA strand, time and/or date stamps, reagent lot numbers, etc.

“CGH” or “Comparative Genomic Hybridization” refers to techniques for identification of chromosomal alterations (such as in cancer cells, for example). Using CGH, ratios between tumor or test sample and normal or control sample enable the detection of chromosomal amplifications and deletions of regions that may include oncogenes and tumor suppressive genes, for example.

“Housekeeping genes” refer to a set or list of genes that are detected by analyzing prior existing data, wherein the data indicates that such genes identified as housekeeping genes remain substantially neutral over all of the data considered. Such housekeeping genes are then applied prospectively in new experiments, as they are also expected to remain substantially neutral in the new experiments and can thus be used as reference values.

“Inert genes” are genes that are used as references, as they are considered to remain substantially neutral for data being considered. Thus, inert genes may refer to genes that are detected as being consistently neutral (i.e., not significantly expressed or inhibited) based upon analysis of the expression data at hand (e.g., across a set of experiments currently being analyzed). “Inert genes” (sometimes also referred to as “constant genes”) may refer to genes which are substantially inert for a specific study. Hence, these genes tend to have “constant” expression levels in the study. The population properties of such genes are constant for all experiments in the study and are therefore useful for normalization purposes. Additionally or alternatively, housekeeping genes may be considered inert genes.

“Communicating” information references transmitting the data representing that information as electrical signals over a suitable communication channel (for example, a private or public network).

“Forwarding” an item refers to any means of getting that item from one location to the next, whether by physically transporting that item or otherwise (where that is possible) and includes, at least in the case of data, physically transporting a medium carrying the data or communicating the data.

A “processor” references any hardware and/or software combination which will perform the functions required of it. For example, any processor herein may be a programmable digital microprocessor such as available in the form of a mainframe, server, or personal computer. Where the processor is programmable, suitable programming can be communicated from a remote location to the processor, or previously saved in a computer program product. For example, a magnetic or optical disk may carry the programming, and can be read by a suitable disk reader communicating with each processor at its corresponding station.

Reference to a singular item, includes the possibility that there are plural of the same items present.

“May” means optionally.

Methods recited herein may be carried out in any order of the recited events which is logically possible, as well as the recited order of events.

All patents and other references cited in this application are incorporated into this application by reference except insofar as they may conflict with those of the present application (in which case the present application prevails).

Systems, methods and computer readable media are provided for labeling samples to be sequenced with unique identifying labels for detection of the labels during the detection processing of the samples themselves. The unique identifiers, once detected, may be used to infer characteristics associated with the samples to which they are attached, respectively.

With the advent of high-throughput sequencing techniques, the present systems and methods provide for labeling samples with unique identifiers which can be sequenced along with the samples that they are attached to, by the same high-throughput technique, during sequencing of the sample itself.

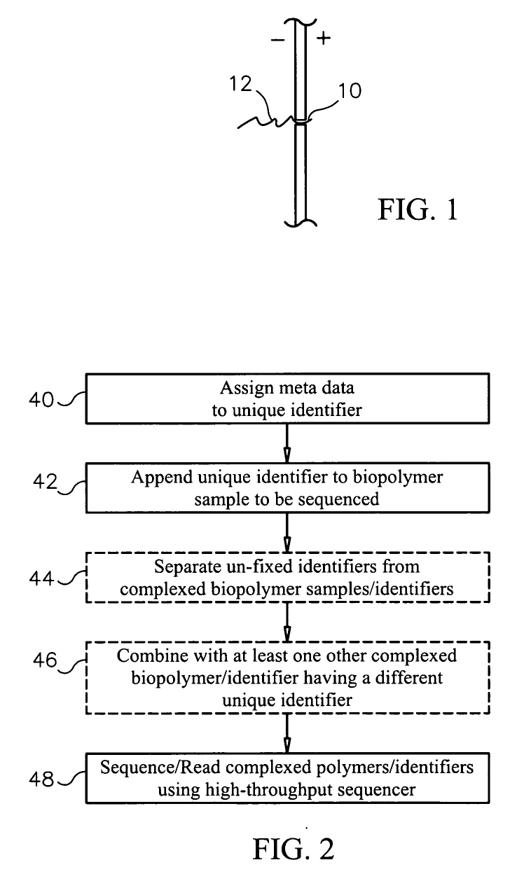

One of the more recent developments in sequencing technology is nanopore sequencing. A nanopore sequencer includes the provision of a very small pore (i.e., nanopore) which may have a diameter in the neighborhood of about 2 nm, for example. An electric field applied across the nanopore (e.g., from the inside of a layer in which the nanopore is situated to the outside of the layer) acts as a driving force that can drive individual nucleic acid molecules to move through the nanopore 10 (see FIG. 1) on a microsecond to millisecond timescale, as reported by Deamer et al., “Nanopores and nucleic acids: prospects for ultrarapid sequencing”, TIBTECH April 2000, Vol. 18, 147-151, which is hereby incorporated herein, in its entirety, by reference thereto. Because the nanopore is so narrow, it is restrictive, and the molecules are translocated through the nanopore as single strands, in strict linear sequence.

As a nucleic acid 12 passes through a nanopore 10 it generates a distinctive electrical signal as it enters and passes through the nanopore 10. One technique for nanopore sequencing relies on the premise that each base in the nucleic acid (i.e., A, C, T and G) will modulate the signal in a specific and measurable way as it passes through nanopore 10. Theoretically, it is reported that sequencing speeds of between one thousand and ten thousand bases per second may be achievable, although these speeds have yet to be attained.

The present methodology would employ nanopore sequencing or some other high throughput sequencing technology to read identifiers attached to nucleic acid sequences in the process or reading or sequencing the nucleic acids themselves. Typically, the identifiers used to tag the nucleic acid sequences would be unique barcode identifiers in the form of an additional unique sequence of nucleic acids appended to the nucleic acid sequence that is being tagged, the barcode being appended by ligation, for example. However, any molecular barcode that is configured to be translocated through a nanopore and that generates a modulated signal to form a unique, repeatable, characteristic signature identifying the barcode as unique from other barcodes may be employed. Other forms of candidates for unique barcodes that may be employed, and are typically charged, include charged block copolymers, examples of which are disclosed in U.S. Publication No. 2002/0142344 A1. For use as barcodes, charged block copolymers may be ligated to respective nucleic acid sequences to be tagged, for example.

As to barcodes formed of unique nucleic acid sequences, there exist several methods for generating extra nucleic acid sequences appended to DNA, where the appended sequence of nucleic acid sequences may be used as a barcode. One method for attaching nucleic acid sequences is taught by U.S. Pat. No. 6,150,516 (Brenner et al.), which is hereby incorporated herein, in its entirety by reference thereto. Brenner et al. teaches an oligonucleotide tag attached to polynucleotides (such as DNA) by polymerase chain reaction (PCR) using primers containing the tag sequence. The term “oligonucleotide” as used herein includes linear oligomers of natural or modified monomers or linkages, including deoxyribonucleosides, ribonucleosides, anomeric forms thereof, peptide nucleic acids (PNAs), and the like, capable of binding to a target polynucleotide by way of regular pattern of monomer-to-monomer interactions, such as Watson-Crick type of base pairing, base stacking, Hoogsteen or reverse Hoogsteen types of base pairing, or the like. Hereinafter, the PCR technique is assumed to be the method for appending tag sequences to DNA. However, it should apparent to those of ordinary skill in the art that other techniques, such as modifications of chemical methods of DNA synthesis disclosed by Pirrung et al, “Comparison of Methods for Photochemical Phosphoramide-Based DNA Synthesis”, Journal of Chemical Physics, 1995, 60, 6270-6276, may be used to add barcodes to the ends of un-amplified DNA without deviating from the present teachings. Pirrung et al, Journal of Chemical Physics, 1995, 60, 6270-6276, is hereby incorporated herein, in its entirety, by reference thereto.

FIG. 2 shows a flow chart of events that may be carried out when sequencing according to an embodiment of the present invention. At event 40, metadata is assigned to a unique identifier that is to be appended to at least one DNA strand that is to be sequenced. “Metadata” may include any information that is useful to track along with the sample/DNA strand. Examples of metadata include, but are not limited to: lab protocols used for the associated sample/DNA strand, time and/or date stamps, reagent lot numbers, etc. Note that there could be multiple instances of a particular sample/DNA strand that a user may want to identify with different individual bar codes. For example, for different instances of the same sample, there may have been different protocols used to prepare the different instances of the sample, of technicians or even labs that were involved in the preparation of the different instances of the samples may be different, and it may be desirable to track this information as associated metadata. The system stores the metadata with some identifying characteristic of the unique identifier, such that the system can readily look up the metadata when the unique identifier is read, identified or sequenced by the high-throughput sequencer to be used. A characteristic signature of the unique identifier, when read by the high throughput sequencer to be used, may be stored along with the metadata for that unique identifier.

At event 42 the unique identifier is appended to DNA strands that are to be sequenced for identification of what is contained in the DNA strands. Note that the DNA strands may be from a particular sample, for example, where all strands may be appended with the same unique identifier. Optionally, known fragmentation processing techniques may be carried out prior to appending the unique identifiers, so as to provide samples having desired characteristics. Alternatives to appending a unique identifier may be optionally carried out at event 42 in order to create a unique identifier associated with the sample (e.g., processing with restriction enzyme, etc.), as described in more detail below.

After processing to complete attachment of the identifiers to the strands to an extent considered to be sufficient to attach identifiers to all strands (which may include various incubation techniques and times that will vary depending upon the type of identifiers being attached, or which may include other techniques, such as “growing” the identifiers, etc.), then a separation of any unbound identifiers from the mixture including the DNA strands complexed to identifiers may be carried out at event 44, if desired, although this is typically not carried out. It is not necessary to separate unbound identifiers, since any unbound identifiers or unbound sample that are read for identification can be simply ignored as not including the requisite sample plus appended unique identifier. However, if a user decides to remove unbound identifiers, one technique for doing so is to immobilize the sample strands by providing complementary probes on a surface (such as a microarray, for example, or beads) which, in turn, immobilizes the identifiers that are bound to the strands. The unbound identifiers can then be removed by a washing or rinsing step. Various techniques may be applied to perform such a separation, which may vary, depending upon the type of identifier used, but which are also generally known in the art.

After separation (if desired), the complexed DNA/identifier strands are ready to be sequenced by a high throughput sequencer at event 48. It is indicated at event 46, that the complexed DNA/identifier strands may be combined with at least one other complexed strand/identifier that has a different unique identifier than those currently appended to the strands in the current round of processing events described above. For example, if a first sample is tagged with a first unique barcode, and a second sample is tagged with a second unique barcode, then these samples can actually be mixed together for multiplex sequence processing of both samples in a single run. There is no concern regarding contamination (assuming, of course, that the samples are not somehow reactive with one another), since each strand read/sequenced, will also have its unique identifier read/sequenced so that the system can automatically identify from which sample the sequenced strand originated, by referencing the metadata associated with the identifier that was read, This can greatly improve throughput speed of sequencing processing, while also relieve somewhat the very strict requirements for prevention of cross-contamination. That is, users may mix several samples together and process them through a single, high, throughput sequencer, or enhance efficiency even further by feeding multiple high-throughput sequencers in parallel with a container holding a mixture of samples.

A single sample may be advantageously processed in parallel by multiple high-throughput processors as well. Additionally, at the end of processing one sample, the system is set up to record sequencing information for the next sample, identified by the next unique identifier. Thus, the user/processor does not have to be concerned with any residue remaining in the system from the first sample, since if a sequencer reads any of the first sample while processing the second sample, the system will identify each first sample read by the unique identifier. Since it will not match the unique identifier for the second sample, the system will simply ignore this sequence. Likewise, if a sequence is read that does not contain any identifier, the system will not know whether that sequence belongs to the present sample or some other previous sample and will therefore disregard that sequence. The same is true during multiplex processing, since the system does not know which sample that the sequence with no identifier belongs to.

Thus, for very high-throughput scenarios, tagging each sample sequence can reduce risks of cross-contamination even when samples are not multiplexed, as any sequence that is not properly barcoded, or has a non-relevant barcode, can be ignored in the sequence analysis of the high-throughput instruments. Operators of the instruments need not be concerned about residual contamination from previous samples remaining in the system, because any such sequence will either have no barcode or an incorrect barcode and can be eliminated from consideration.

For barcoded strands where the barcode is a unique sequence of nucleic acids (described further below), a high-throughput sequencer such as a nanopore device may sequence the barcode in the same way that the sample stand is sequenced, i.e., base-by-base. One well-known technique suitable for generating an extra sequence to be appended to DNA is referred to as the “tailed-primer PCR” technique. Using this technique, PCR (polymerized chain reaction) primers are created for DNA amplification. However, in addition to the prime sequence, an additional 5′ “tail” of bases may be added for some purpose. One such purpose may be as a self-probing amplicon, see Whitcombe et al., “Detection of PCR products using self-probing amplicons and fluorescence.”, Nat. Biotechnol. 1999 August; 17(8:804-7, which is hereby incorporated herein, in its entirety, by reference thereto.

Using techniques to create molecular barcodes using nucleic acids, primers that have tails of a specific barcode sequence will produce amplicons with these barcodes at the ends of the sequence. Either 3′ or 5′ labeled amplicons may be produced, or sequences may be produced where both ends contain the same or different barcodes. Since the bases A,C,T and G enable a simple four letter alphabet that can be used to encode data, barcodes can be created for unique identification of the material to which the barcode is attached. To aid in subsequent reading and analysis of such barcodes, suitable stop/start markers (e.g., a unique sequence of bases (A,T,C and G) that can be pattern-matched by the system during sequencing, wherein the sequencing of the start or stop sequence is identified by mating it to the same sequence as stored by the system. Such start and stop sequences should be chosen to be non-homologous to any expected sequence (e.g., in the sample) to avoid mistaken identification of a start or stop marker somewhere within a sample sequence being read. Thus by constructing a unique sequence of stop/start markers and appending it to a sample, further information can be carried, stored and/or pointed to with regard to that sample upon identification of the sample via reading of the unique sequence. Thus, start and stop markers may be created to facilitate location and reading of the barcodes and distinguish properly barcoded sequences from sequences lacking barcodes. Further such tailed primers may be targeted for specific sequences of interest (e.g., coding regions, SNP's, CGH break points, etc.) or suitably tailed random primers may be used to amplify less specifically.

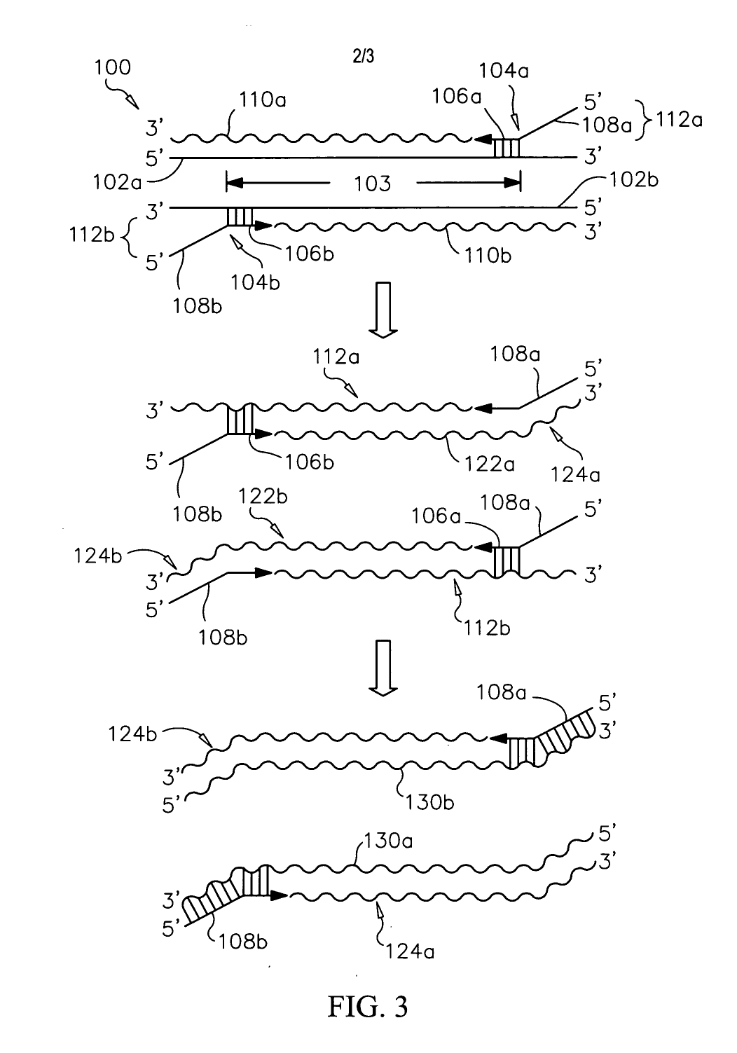

Referring now to FIG. 3, a schematic diagram 100 illustrates steps that may be performed for bi-directional sequencing of PCR products using tailed-primers in accordance with one embodiment of the present invention. As illustrated in FIG. 3, two strands 102a-b of a target DNA may include a region 103 of particular interest that a researcher wishes to study, and therefore the researcher wishes to barcode and amplify that region. The selected region 103 may be a specific portion of interest (such as coding regions, single nucleotide polymorphisms (SNPs) or comparative genomic hybridization (CGH) break points) or an entire sequence of the original target DNA. Typically, DNA has two strands and may be separated into two DNA strands 102a-b by a brief heat treatment.

Each of tailed-primers 104a-b may comprise two nucleotide sequences forming one oligonucleotide sequence; PCR part 106 and tail 108. PCR parts 106a-b (shown as arrows) may be synthesized based on the known parts of selected region 103. In some applications, PCR part 106a-b may be randomly sequenced to amplify less specifically. Tail 108a may be appended to the 5′-end of forward PCR part 106a, while tail 108b may be appended to the 5′-end of reverse PCR part 106b. In one embodiment, tail 108 may have a standard sequence, such as M13, T7 or T3. In another embodiment, each of tails 108a-b may be designed to implement stop/start markers. In both embodiments, as will be explained later, tail 108 may correspond to a barcode that may be used to identify the DNA to which tail 108 is appended.

Initial synthesis of newly formed DNA sequences 112a-b may be primed from the PCR parts 106a-b on original target strands 102a-b. As mentioned, a brief heat treatment may be required to separate original target strands 102a-b from each other. A subsequent cooling of original target strands 102a-b in the presence of large excess of tailed-primers 104 may allow these tailed-primers 104a-b to hybridize to the original target strands 102a-b. The annealed mixture may be incubated with DNA polymerase and an abundance of the four nucleotides (A, C, T, and G), so that the downstream region 110 of PCR part 106 may be selectively hybridized. Thus, upon completion of the first step, each synthesized DNA strand 112 may include a tailed-primer 104 and synthesized sequence 110 indicated by a wavy line.

In the second step, synthesized DNA strands 112a-b may become templates for intermediate synthesized DNA strands 124a-b. DNA 124a may include tailed-primer 104b and synthesized sequence 122a. The synthesized sequence 122a (shown as a wavy line) may be primed from another reverse PCR part 106b and hybridized to the 5′-end of the tail 108a. Likewise, synthesized DNA 124b may include a tailed-primer 104a and synthesized sequence 122b, where synthesized sequence 122b may be primed from a forward PCR part 106a and hybridized to the 3′-end of tail 108b.

Still referring to FIG. 3, intermediate DNA strands 124a-b may become templates for synthesizing barcoded DNA strands 130a-b in the third step. Each barcoded DNA 130 may include a copy of selected region 103 of corresponding original target DNA strand 102 and two barcodes that correspond to tails 108a-b. In an alternative embodiment, one of tailed-primers 104a-b may not have the PCR part. In this embodiment, barcoded DNA 130 may have only one barcode sequence appended to the copy of selected region 103 of corresponding original target DNA strand 102. By repeating the heating and annealing cycles, barcoded DNA strands 130a-b may be amplified to generate sufficient population.

The ability to identify a barcode as a unique nucleotide sequence may also be enhanced by using synthetic DNA/RNA analogues (SNA) rather than using naturally occurring DNA. SNA's are well-known in the art and are used for a variety of purposes. Analogues may be created by modifying various structural elements of natural nucleic acids.

Further, SNA's may be designed/carefully chosen so as to have different electrical characteristics, relative to one another, as well as to the bases A,T,C and G, such that when these SNA/s pass through a nanopore sequencer, they are detected and distinguishable by the detected electrical signal, from A,T,C or G or any other SNA that may be currently being used in a procedure. Such SNA's may be used to delimit a barcoded region (to delimit a barcode), or an SNA may be used to form a barcode itself, by forming a sequence that is distinguishable from the naturally occurring sequence. However, care should be taken to ensure that the synthetic modifications do not increase the size of the SNA to the extent that it is no longer capable of traversing through a nanopore. Further the electrical characteristics of each SNA need to be distinguishable from naturally occurring nucleic acids when sequences are read, as noted above.

Ideally barcodes should not have any homology to any sequence that is likely to be read during sequencing. In order to reduce the chances that a naturally occurring fragment end (from fragmented DNA) matches a barcode sequence, one can attempt to choose barcode sequences that are non-homologous to the organism to be studied. Alternatively, only one unique sequence (e.g., a single unique sequence) need be determined or used if used as a delimiter. The probability any given sequence will have no homology to any sequence in samples from an organism with which it will be associated can be greatly increased by checking such sequence using BLAST or some similar database searching tool to check the purported unique sequence against know sequences in the organism from which tissue samples will be taken to be associated with the unique sequence. When used as a delimiter, the single unique sequence may be employed to delimit both ends of that portion that makes up the unique identifier. Since, when sequencing a strand, the single unique sequence will always be read prior to reading the unique identifier that is delimited on both ends by the single unique sequence, the unique identifier in this case need only be unique as to identification of the sample that it is appended with, and does not need to be non-homologous with all sequences of the sample tissue.

Advantageously, only the one unique sequence (single unique sequence) need be distinct from any sequence from the organism likely to be read and the same unique sequence/single unique sequence can be used to delimit all barcode sequences used. The sequences for the barcodes, on the other hand, can be freely chosen (e.g., non-homologous) without regard to whether any particular sequence is likely to match a sample sequence, because during reading, it will already be known when a barcode is being read, regardless of its content, because the unique sequence/single unique sequence alerts the reader to this fact. During sequencing the barcodes may be detected by scanning the sequences for the barcode delimiters (unique sequence/single unique sequence) and extracting the barcodes from the sequences in the areas located between the delimiters.

Another alternative approach to providing identifiable sequence labeling involves digesting DNA samples with enzymes that cleave the samples at specific target sequences. Restriction enzymes are examples of such enzymes. A number of different restriction enzymes are currently known that each cleave at different, very specific, known recognition sites. Accordingly, the ends of digested fragments that result from such a digestion each have a characteristic sequence that depends upon the particular enzyme that was used to perform the digestion. Thus by carefully examining the ends of any sequence read by a sequencer, the characteristic end sequence will directly identify the particular enzyme that was used to digest that sequence having just been read. Therefore, if different samples are digested by different enzymes, each having a distinct recognition site, then the enzymes used can be identified in the manner just described, which in turn identifies the particular sample that the sequence belongs to, since a record is retained of which enzymes were used to digest which samples. Of course, if no characteristic sequence is read while reading any given sequence, this particular sequence will be discarded since it cannot be determined which sample it originated from.

For example, the target 5′-3′ sequence for the enzyme Hpa I is “GTTAAC” and cleaves between the T and A bases. Thus when digesting with Hpa I restriction enzyme, the resultant fragments of a sample strand digested would have characteristically identifiable ends “ . . . GTT” and “AAC . . . ”. In contrast, the enzyme Sma I cleaves the sequence “CCCGGG” between the C and G bases, leaving characteristic fragment ends “ . . . CCC” and “GGG . . . ”. Thus by noting the final three bases of any fragment read during sequencing, it can be determined which enzyme was used to perform the digestion. Further, if one sample was treated with Hpa I and another sample was treated with Sma I, then the source sample itself, from which the fragment originated, can also be readily identified by noting the final three bases of the fragment read. Use of enzymes to digest samples as described provides the benefit that barcodes do not have to be ligated to the samples being sequenced, thereby eliminating a processing step as compared to other barcode schemes. Further, the digestion reduces the DNA strand lengths which may be beneficial when sequencing with a nanopore sequencer, as relatively shorter length strands may be easier to pass through a nanopore.

A barcoded DNA sample, such as prepared in accordance with the steps of FIG. 3, for example, may be sequenced by a high-throughput sequencer, such as a nanopore device. Once a molecule destined for sequencing is so labeled, a nanopore device can easily read off the barcode tag as part of the sequence and thereby the system may associate the sequence with whatever metadata is associated with the barcode, as noted above. When performing multiplex processing, one of the metadata identified by the barcode is the sample from which the molecule was derived. Therefore, no matter how many samples are mixed in the same batch/run, each sequence may be uniquely identified with the source of the material and the multiplexed samples can thusly be easily de-convoluted.

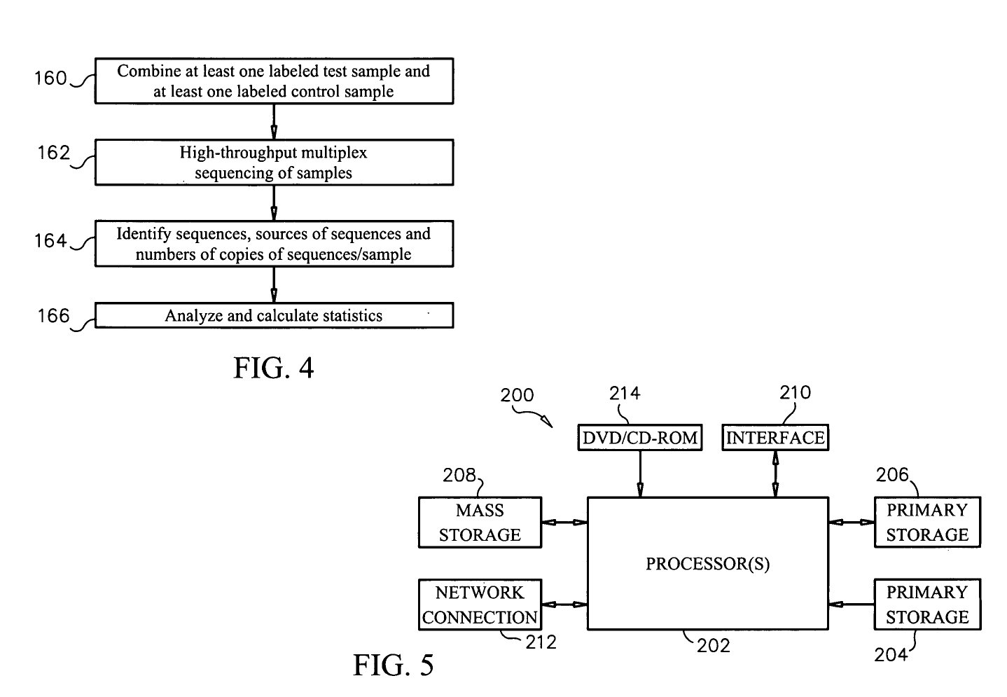

The present techniques may also be applied to perform ratio-based abundance analysis (of CGH or Gene Expression values, for example), by analyzing a test versus a control sample in the same run. Of course, more than one test sample may be included in the run, as well as more than one control sample if desired. Referring to FIG. 4, after appropriately labeling each sample with a unique identifier (such as a barcode), in a manner as described herein, at event 160, the sequences are identified by running them through a high-throughput sequencer according to a multiplex sequencing scheme as described herein, e.g., sequences may be run through a single sequencer, or run in parallel through a plurality of sequencers, which can be coordinated with a system processor for assignment of metadata correlated with the identified barcodes, and correlating this with the information contained in the sequences.

In addition to identifying the sequences and the sources of the sequences (i.e., test or control sample), the system in this example also keeps a count of the copy numbers of each sequence at event 164, which counts are also correlated with source (test sample or control sample). After significant numbers of sequences have been read/sequenced (i.e., the run is sufficiently long to render the counts statistically significant), ratios of the copy numbers, between the test sample and the control sample may be calculated by the system at event 166. Optionally, further statistical processing of the counts and/or ratios may be performed by the system, such as statistical treatments that are currently applied in CGH analysis. By running the test and control samples together according to the multiplex techniques, systematic experimental errors are reduced, since both the test and control samples experience the same environmental and systematic conditions as they are sequenced.

Further, using a PCR method as described above, select sequences of interest may be amplified and probed, rather than the whole genome. Using this approach, high-throughput sequencing can be applied to perform many of the same measurements as DNA microarrays as well as other sequence-based assays. For example, a first unique identifier may be appended to sequences (in a manner as described above) in a test sample and a second unique identifier may be similarly appended to sequences in a control sample. Test samples and corresponding control samples for such measurements may be come from a wide variety of sources. Non-limiting examples of test and control samples include: diseased tissue sample versus normal tissue sample, treated (such as by a drug or some other chemical and/or physical treatment) versus untreated tissue sample, aggressive tissue versus non aggressive tissue sample, tissue/cells responding to treatment versus tissue/cells not responding to treatment, etc.

Using the present system, a ratio between the number of test sample biopolymers identified/sequenced and the number of control sample biopolymers identified/sequenced may be calculated. By mixing together the complexed sequences of the test sample sequences and appended first unique identifiers with complexed sequences of the control sample sequences and second unique identifiers, and randomly passing the complexed sequences from the mixture through at least one high-throughput sequencer, the sequences and their associated identifiers are read (e.g., sequenced or identified). By counting or tracking the number of identical sequences for each different sequence and relative to their origins (test or control sample), comparisons can then be made as to the number of occurrences of any particular sequence in the test sample and in the control sample, respectively. From such a comparison, a ratio can be calculated, similar to an expression ratio. Typically, equal amounts of the test sample and control sample are mixed, each at the same concentration, as this makes ratio calculations more straightforward. However, measurements may still be carried out when the amounts and/or concentrations of test and control samples are unequal, as it may be possible to normalize the data. For example, by tracking inert or housekeeping genes, the numbers of which are not expected to vary between the test sample and the control sample, the calculated ratio of the observed inert genes in the test sample to the observed inert genes in the control can be adjusted to the expected ratio of one-to-one. All other measurements for other genes can then be adjusted proportionately to normalize the ratios. Further, other known normalization techniques that are practiced for normalizing gene expression ratios from microarrays may also be applied to the present techniques. Such normalization techniques include, but are not limited to, normalization based upon inert or housekeeping genes, spike-in controls, and/or centering means.

Even when equal amounts of the test sample and control sample are mixed, each at the same concentration, not all copies of the strands in each sample are likely to be labeled (i.e., one hundred percent labeling of the samples is not likely to be achieved), and thus the ratios from these analyses may also need to be further statistically processed for the likelihood that not all sequences were labeled. However, there should not be bias in this regard, since both the control and test samples should have the same likelihood to have identifiers append to the strands thereof. Further any sample used will contain a very large number of cells so that a large count number of any sequence included in the sample is expected to be measured/identified. Therefore by simply collecting sequence counts over comparable periods of time to see which sample gives more copies than others (if any) can identify CGH ratios. Similarly, for expression ratio measurements a statistically significant number of copies of any particular mRNA representing expression of a particular gene need be measured with regard to both test and control samples. Using the techniques described, the present invention may be used for CGH measurements, mRNA expression ratio measurements, SNP measurements, or to measure any other sequence-based assay. Furthermore, multiple experiments may be measured by multiplexing as described, wherein more than one test sample may be measured against the same or different control samples, all from the same mixture, for example.

FIG. 5 illustrates a typical computer system in accordance with an embodiment of the present invention. The computer system 200 may include any number of processors 202 (also referred to as central processing units, or CPUs) that are coupled to storage devices including the first primary storage 204 (typically a random access memory, or RAM), and the second primary storage 206 (typically a read only memory, or ROM). As is well known in the art, the first primary storage 204 acts to transfer data and instructions uni-directionally to the CPU and the second primary storage 206 is used typically to transfer data and instructions in a bi-directional manner. Both of these primary storage devices may include any suitable computer-readable media such as those described above. A mass storage device 208 is also coupled bi-directionally to CPU 202 and provides additional data storage capacity and may include any of the computer-readable media described above. Mass storage device 208 may be used to store programs, data and the like and is typically a secondary storage medium such as a hard disk that is slower than primary storage. It will be appreciated that the information retained within the mass storage device 208, may, in appropriate cases, be incorporated in standard fashion as part of primary storage 206 as virtual memory. A specific mass storage device such as a CD-ROM 214 may also pass data uni-directionally to the CPU.

CPU 202 is also coupled to an interface 210 that includes one or more input/output devices such as such as video monitors, track balls, mice, keyboards, microphones, touch-sensitive displays, transducer card readers, magnetic or paper tape readers, tablets, styluses, voice or handwriting recognizers, or other well-known input devices such as, of course, other computers. Finally, CPU 202 optionally may be coupled to a computer or telecommunications network using a network connection as shown generally at 212. With such a network connection, it is contemplated that the CPU might receive information from the network, or might output information to the network in the course of performing the above-described method steps. The above-described devices and materials will be familiar to those of skill in the computer hardware and software arts.

The hardware elements described above may implement the instructions of multiple software modules for performing the operations of this invention. For example, instructions for interpreting signals, the voltages of which vary with differing bases being represented, may be stored on mass storage device 208 or 214 and executed on CPU 208 in conjunction with primary memory 206.

In addition, embodiments of the present invention further relate to computer readable media or computer program products that include program instructions and/or data (including data structures) for performing various computer-implemented operations. The media and program instructions may be those specially designed and constructed for the purposes of the present invention, or they may be of the kind well known and available to those having skill in the computer software arts. Examples of computer-readable media include, but are not limited to, magnetic media such as hard disks, floppy disks, and magnetic tape; optical media such as CD-ROM, CDRW, DVD-ROM, or DVD-RW disks; magneto-optical media such as floppy disks; and hardware devices that are specially configured to store and perform program instructions, such as read-only memory devices (ROM) and random access memory (RAM). Examples of program instructions include both machine code, such as produced by a compiler, and files containing higher level code that may be executed by the computer using an interpreter.

While the present invention has been described with reference to the specific embodiments thereof, it should be understood by those skilled in the art that various changes may be made and equivalents may be substituted without departing from the true spirit and scope of the invention. For example, other methods for appending barcode sequences to DNA may be substituted, e.g., such as using phosphoramidite chemistry as described in Pirrung et al., “Comparison of method for photochemical phosphoramidite-based DNA synthesis”, which was incorporated by reference above. In addition, many modifications may be made to adapt a particular situation, material, composition of matter, process, process step or steps, to the objective, spirit and scope of the present invention. All such modifications are intended to be within the scope of the claims appended hereto.

That which is claimed is:

1. A method of sequencing a biopolymer specimen and tracking a source from which the specimen was derived, said method comprising the steps of:
processing the biopolymer specimen to provide a unique identifier with the biopolymer specimen as processed, wherein said unique identifier represents metadata identifying a source sample from which the biological specimen was taken, and said unique identifier is configured to form a unique, repeatable, characteristic signature when read by a high-throughput sequencer;
passing the biopolymer specimen including the unique identifier through the high-throughput sequencer and identifying a sequence of the biopolymer specimen, as well as identifying the unique identifier as each passes through the high-throughput sequencer; and
correlating the identified sequence of the biopolymer specimen with the source sample from which the identified sequence was derived, based upon the identifier metadata derived from said identification of the unique identifier for that respective sequence.

2. The method of claim 1, wherein said high-throughput sequencer comprises a nanopore device.

3. The method of claim 1, wherein said unique identifier comprises a barcode including a unique sequence of nucleic acid bases, and wherein said processing comprises appending said barcode to the biopolymer specimen.

4. The method of claim 1, wherein the biopolymer specimen comprises a DNA strand.

5. The method of claim 1, wherein the biopolymer specimen comprises an RNA strand.

6. The method of claim 3, wherein said unique sequence of nucleic acid bases comprises SNA.

7. The method of claim 1, wherein said processing comprises digesting said biopolymer specimen with a specific restriction enzyme, and wherein said unique identifier comprises nucleic acid bases resulting from the specific restriction enzyme digest.

8. The method of claim 1, wherein said unique identifier comprises a sequence of nucleic acid bases and said unique identifier is delimited by a unique sequence that is non-homologous to said biopolymer specimen.

9. A method for multiplex sequencing biopolymer samples, said method comprising the steps of:
processing biopolymer strands in a first biopolymer sample to provide a first unique identifier with each said biopolymer strand so processed, wherein said first unique identifier includes metadata identifying said first biopolymer sample, and said first unique identifier is configured to form a unique, repeatable, characteristic signature when read by a high-throughput sequencer;
processing biopolymer strands in a second biopolymer sample to provide a second unique identifier with each said to biopolymer strand in said second biopolymer sample so processed, wherein said second unique identifier includes metadata identifying said second biopolymer sample, and said second unique identifier is configured to form a unique, repeatable, characteristic signature different from the signature formed by said first unique identifier, when read by the high-throughput sequencer;
mixing together processed strands of said first biopolymer sample with processed strands of said second biopolymer sample;
randomly passing at least one processed strand through at least one high-throughput sequencer and identifying the strand sequence, as well as identifying the unique identifier as each processed strand passes through the high-throughput sequencer; and
correlating the identified sequences of the biopolymers with the samples from which they were derived, based upon the identifier metadata derived from said identification of the unique identifier for that respective biopolymer strand.

10. The method of claim 9, further comprising processing biopolymer strands in at least one additional biopolymer sample to provide an additional unique identifier for each additional biopolymer sample, respectively, wherein each unique identifier associated with each additional biopolymer sample is unique from all other unique identifiers associated with all other additional biopolymer samples and from said first and second unique identifiers, and wherein processed strands from each additional biopolymer sample are mixed, randomly passed, identified and correlated along with said processed strands from said first and second biopolymer samples.

11. The method of claim 9, wherein at least one of said first and second biopolymer samples comprises DNA strands.

12. The method of claim 9, wherein at least one of said first and second biopolymer samples comprises RNA strands.

13. The method of claim 9, wherein at least one of said first and second unique identifiers comprises a barcode including a unique sequence of nucleic acid bases, and wherein said processing comprises appending said barcode to the biopolymer strand from said respective biopolymer sample.

14. The method of claim 13, wherein said unique sequence of nucleic acid bases comprises SNA.

15. The method of claim 9, wherein at least one of said processing biopolymer strands in said first sample and processing biopolymer strand in said second sample comprises digesting said biopolymer strands in said respective sample, with a specific restriction enzyme, and wherein said unique identifier comprises nucleic acid bases resulting from the specific restriction enzyme digest.

16. The method of claim 9, wherein at least one of said first and second unique identifiers each comprise a sequence of nucleic acid bases that is unique from the other and each said unique identifier is delimited by a unique sequence that is non-homologous to said biopolymer specimen.

17. A method of sequencing biopolymeric specimens through a high-throughput sequencer, said method comprising the steps of:
processing sequences in a first biopolymeric sample to provide a first unique identifier with each processed sequence, wherein said first unique identifier represents metadata identifying said first biopolymeric sample, and said first unique identifier is configured to form a unique, repeatable, characteristic signature when read by a high-throughput sequencer;
passing the sequences having first unique identifiers associated therewith through the high-throughput sequencer and identifying each sequence of the first biopolymeric sample as well as identifying the first unique identifier associated therewith, as each passes through the high-throughput sequencer;
correlating the identified sequences with the first biopolymeric sample from which the identified sequences were derived, based upon the identifier metadata derived from said identification of the first unique identifier for each respective sequence;
processing sequences in a second biopolymeric sample to provide a second unique identifier with each processed sequence from said second biopolymeric sample, wherein said second unique identifier represents metadata identifying said second biopolymeric sample, and said second unique identifier is configured to form a unique, repeatable, characteristic signature when read by a high-throughput sequencer;
passing the sequences having second unique identifiers associated therewith through the high-throughput sequencer and identifying each sequence, as well as identifying the second unique identifier associated therewith, as each passes through the high-throughput sequencer; and
correlating the identified sequences associated with the second unique identifiers with the second biopolymeric sample from which the identified sequences were derived, based upon the identifier metadata derived from reading said second unique identifier for each respective sequence, but ignoring the identified sequences when the associated unique identifier read is not the second unique identifier, or there is no unique identifier associated with the sequence.

18. The method of claim 11, wherein at least one of said first and second biopolymer samples comprises DNA strands.

19. The method of claim 11, wherein at least one of said first and second biopolymer samples comprises RNA strands.

20. A method of sequencing biopolymeric specimens through a high-throughput sequencer, said method comprising the steps of:
processing sequences in at least one biopolymeric sample to provide a unique identifier with each said sequence so processed, wherein said unique identifiers with respect to each sample are unique from unique identifiers with respect to all other samples and each said unique identifier represents metadata identifying said biopolymeric sample from which each sequence associated with each said unique identifier was taken, and each said unique identifier is configured to form a unique, repeatable, characteristic signature when read by a high-throughput sequencer;
passing the sequences having associated unique identifiers through the high-throughput sequencer and identifying each sequence, as well as identifying any unique identifier associated therewith, as each passes through the high-throughput sequencer; and
correlating the identified sequences with the respective biopolymeric samples from which the identified sequences were derived, based upon the identifier metadata derived from said identification of the associated unique identifier for each respective sequence, but ignoring the identified sequences when the associated unique identifier read is not a unique identifier associated by said processing step, or there is no unique identifier associated with the sequence.

21. A method of performing ratio-based analysis with a high throughput sequencer, said method comprising the steps of:
processing sequences in a test biopolymeric sample to associate a first unique identifier with each sequence so processed, wherein said first unique identifier represents metadata identifying said test biopolymeric sample, and said first unique identifier is configured to form a unique, repeatable, characteristic signature when read by a high-throughput sequencer;
processing sequences in a control biopolymeric sample to associate a second unique identifier with each sequence from the control sample so processed, wherein said second unique identifier represents metadata identifying said control biopolymeric sample, and said second unique identifier is configured to form a unique, repeatable, characteristic signature different from the signature formed by said first unique identifier, when read by the high-throughput sequencer;
mixing together processed sequences of said test biopolymeric sample associated with said first unique identifiers, with processed sequences of said control biopolymeric sample associated with said second unique identifiers;
randomly passing processed sequences through at least one high-throughput sequencer and identifying the sequences, as well as identifying the unique identifiers associated therewith as the processed sequences pass through a high-throughput sequencer, respectively;
correlating the identified sequences with the samples from which they were derived, based upon the identifier metadata derived from said identification of the unique identifier associated with that respective sequence;
counting the number of times that a particular sequence is read with regard to said first and second unique identifiers; and
calculating a ratio comparing the number of times that the particular ratio was identified as associated with said first and second identifiers, respectively.

22. The method of claim 21, further comprising processing biopolymer sequences in at least one additional biopolymeric sample to associate a unique identifier with each said sequence so processed from each said additional biopolymeric sample, wherein each unique identifier associated with each additional biopolymeric sample is unique from all other unique identifiers associated with all other additional biopolymeric samples and from said first and second unique identifiers, and wherein processed sequences from each additional biopolymeric sample are mixed, randomly passed, correlated, counted and ratio-calculated against at least one other biopolymeric sample along with said processed sequences from said first and second biopolymeric samples.

23. The method of claim 22, wherein said counting and calculating steps are carried out with regard to at least one additional particular sequence different from said particular sequence.

24. The method of claim 21, wherein said biopolymeric samples are DNA samples.

25. The method of claim 21, wherein said biopolymeric samples are RNA samples.

26. The method of claim 21, wherein said ratio-based abundance analysis comprises CGH analysis.

27. The method of claim 21, wherein said ratio-based abundance analysis comprises gene expression analysis.

28. The method of claim 21, wherein said ratio-based abundance analysis comprises SNP analysis.
